(19) United States
(12) Patent Application Publication
(10) Pub. No.: US 2024/0041364 A1
(43) Pub. Date: Feb. 8, 2024

(54) REFERENCE ELECTRODE & ION SELECTIVE MEMBRANE

(71) Applicant: University of Southampton, Southampton (GB)

(72) Inventors: Roeland Hubertus Gerardus MINGELS, Southampton, Hampshire (GB); Hywel MORGAN, Nomansland, Salisbury, Wiltshirr (GB)

(21) Appl. No.: 18/265,580

(22) PCT Filed: Nov. 25, 2021
(86) PCT No.: PCT/GB2021/053065

§ 371 Date: Jun. 6, 2023

(30) Foreign Application Priority Data
Dec. 7, 2020 (GB) 2019249.8

(51) Int. Cl. A61B 5/1473; G01N 27/333; G01N 27/30

(52) U.S. Cl. CPC A61B 5/1473; G01N 27/3335; G01N 27/301; A61B 2503/40; A61B 2562/125

(57) ABSTRACT

An integrated reference electrode for use in an electrochemical measurement system, comprising: a reference electrode in combination with a hygroscopic hydrogel electrolyte impregnated and contained in a porous framework, wherein the hygroscopic hydrogel electrolyte is adapted to contact the reference electrode when in a hydrated state. Also, an ion selective membrane with an aromatic epoxy polymer made from one or more monomers having at least one epoxide group, and a chloride salt or a silver—silver chloride physically trapped in the aromatic epoxy polymer. On contact with water the aromatic epoxy polymer forms a network of channels for ion exchange. The membrane may be used between an electrolyte and an external fluid environment; or the membrane may be coated upon the surface of a reference electrode.

### FIELD OF THE INVENTION

The present invention relates to an integrated reference electrode, a method of manufacturing an integrated reference electrode, and an ion selective membrane.

### BACKGROUND OF THE INVENTION

A reference electrode is an electrode that provides a stable reference potential for electrochemical potentiometric and amperometric measurements. The reference electrode can be used as a half cell in an electrochemical cell against which the potential of a working electrode is measured or set. Reference electrodes are ideally insensitive to the target species sensed at the working electrode and thus provide a quantifiable reference potential for changes occurring at the working electrode.

In some conventional reference electrodes, the reference electrode (e.g. commonly a Ag/AgCl electrode) and a filling liquid (commonly 3M KCl) are housed in a tube or other housing (commonly formed of glass or plastic). Ionic contact between the reference electrode and a test solution is established by a porous ceramic frit or ion exchange membrane so as to complete the electrochemical cell formed by the reference electrode and the working electrode. The frit has two main functions: firstly to contain the electrolyte whilst ensuring there is ionic contact (i.e. an exchange of ions across the frit); and secondly to limit the diffusion and mixing of the internal solution with the external environment.

However, conventional reference electrodes face a number of challenges. For instance, any changes in the concentration of the solution (e.g. through diffusion or evaporation, or through osmosis or active transport when implanted into a patient) will affect the potential established by the reference electrode. The use of liquid based electrolytes also poses manufacturing and mechanical challenges, particularly as the electrodes are scaled down for use as implantable sensor systems.

Smaller electrodes also face challenges relating to stability due to the limited amount of silver chloride, or other active coating materials. According to the Nerst-equation, the potential of the Ag/AgCl electrode is determined by the ratio of Ag to AgCl and the concentration of the chloride ion within the surrounding environment. Hence, any changes in either the composition of the electrode layer (i.e. Ag/AgCl) or the chloride concentration affect the potential stability of the reference electrode.

Furthermore, epithelial interfaces absorb excess liquid and ionic species over time through osmotic differences, and this can eventually breakdown the ionic bridge between the working electrode and reference electrode, especially when they are disposed at a distance away from each other (i.e. on separate sensor chips, or disposed on the front and back of a substrate). Existing solutions aimed at tackling one or more of these problems include solid state electrolytes, which can be difficult to integrate into multifaceted sensor systems, and quasi-solid polymers (e.g. hydrogels), which still face the persistent problem of evaporation and dehydration and therefore may require storage in highly humid or liquid conditions.

Sterilisation of medical devices incorporating a reference electrode, or other similar device, can also be challenging. Many sterilisation methods are available for such devices, such as autoclaving, irradiation, or gas sterilisation. However, autoclaving degrades hydrogel based systems due to the high temperatures involved, and can compromise the structural integrity of an assembly due to the expansion of any liquids and air trapped in the assembly. Sensors that can use biorecognition elements (e.g. antibodies) as their sensing principle are also irreparably damaged by the high temperatures. Furthermore, irradiation (e.g. by e-beam or γ-rays) damages the electronics, whilst gas sterilisation (e.g. using ethylene oxide—EtO) is less damaging but can leave toxic compounds (such as ethylene glycol and ethylene chlorohydrin) that require removal before reuse. Removal can be performed by leaching of the gas, however this is a slow process, which is costly for process optimisation and validation, as well as supply line and manufacturing times. Any liquids or hydrogels also suffer from dehydration during this process.

### SUMMARY OF THE INVENTION

A first aspect of the invention provides an integrated reference electrode for use in an electrochemical measurement system, comprising: a reference electrode in combination with a hygroscopic hydrogel electrolyte impregnated and contained in a porous framework, wherein the hygroscopic hydrogel electrolyte is adapted to contact the reference electrode when in a hydrated state.

A second aspect of the invention provides a sensor system comprising the integrated reference electrode of the first aspect, and at least one working electrode.

A third aspect of the invention provides a method of manufacturing an integrated reference electrode for use in an electrochemical measurement system so as to provide a reference potential, the method comprising: preparing a hygroscopic hydrogel electrolyte impregnated and contained in a porous framework; providing a reference electrode; and placing the reference electrode and the porous framework having the hygroscopic hydrogel electrolyte therein within a common housing, such that the hygroscopic hydrogel electrolyte is able to contact the surface of the reference electrode when in a hydrated state.

A further aspect of the invention provides a method of manufacturing a sensor system, comprising: manufacturing an integrated reference electrode according to the method of the third aspect; providing a sensor chip having the reference electrode and at least one working electrode, wherein the integrated reference electrode and the sensor chip are housed within the common housing, and the at least one working electrode is in contact with the reference electrode.

Hydrogels are polymeric materials cross-linked to form a three-dimensional network whose hygroscopic properties allow the network to hold a large volume of water. A hygroscopic material is a material that retains and attracts water from its surrounding through absorption.

By providing a hygroscopic hydrogel impregnated and contained in a porous framework, the porous framework provides a structure to support the hygroscopic hydrogel electrolyte such that, when the hydrogel is dried, the hydrogel can be rehydrated at a later point without compromising the hydrogel's ability to maintain good contact with the reference electrode. The porous framework maintains its structure when the hydrogel is dehydrated, ensuring that the hydrogel is well positioned to resume contact with the reference electrode when it is subsequently hydrated. The result is that the typical requirement to maintain the reference electrode in a moist or wet environment at all times, even in storage, is mitigated. The porous framework can also be sterilised due to the absence (or at least reduction in quantity) of liquid in the porous framework. This allows the efficient removal of any toxic compounds introduced during gas sterilization, in particular ethylene oxide.

The integrated reference electrode is a simple and cost effective means of incorporating a biocompatible and hygroscopic electrolyte into an in vivo environment, and overcomes many of the challenges associated with the integration of hydrogel electrolytes in implantable sensor systems.

The reference electrode may be a silver—silver chloride electrode. The reference electrode may be a silver—silver chloride electrode fabricated on a substrate.

The porous framework may be a scaffold, foam or sponge material.

The porous framework may include one or more of poly-urethane (PU) or poly-dimethyl siloxane (PDMS).

The hygroscopic hydrogel electrolyte may be biocompatible and/or bactericidal.

The hygroscopic hydrogel electrolyte may include a chloride salt. The salt may be a potassium chloride salt or sodium chloride salt.

The hygroscopic hydrogel electrolyte may include one or more from the group: acrylamide, alginate, agarose, chitin, chitosan or other chitin derivatives.

The porous framework may be hydrophilic, or the porous framework may have a hydrophilic coating.

The reference electrode may comprise a wire, rod, pellet, or a micro-fabricated electrode.

The integrated reference electrode may further comprise an ion selective membrane outside the porous framework. The ion selective membrane may surround the porous framework.

The reference electrode may be bonded to the porous framework.

The sensor system may further comprise a sensor chip having the reference electrode and the at least one working electrode mounted on the chip, wherein the integrated reference electrode and the sensor chip are housed within a common housing.

The at least one working electrode may be in contact with the hygroscopic hydrogel electrolyte.

The chip may have a first side having the reference electrode and a second side opposite the first side. The chip may provide an open liquid junction between the hygroscopic hydrogel electrolyte on the first side of the chip and an external environment on the second side of the chip.

The sensor system may be an implantable sensor system for implanting in a human or animal body. The sensor system may be an intrauterine sensor system.

The method of the third aspect may further comprise: dehydrating the hygroscopic hydrogel electrolyte within the porous framework before placing in the common housing, and rehydrating the hygroscopic hydrogel electrolyte when in contact with the reference electrode in the common housing.

The method of the third aspect may further comprise: bonding the porous framework having the hygroscopic hydrogel electrolyte therein to the surface of the reference electrode.

The method of the third aspect may further comprise: fabricating the porous framework having the hygroscopic hydrogel electrolyte onto the surface of the reference electrode.

The method of the third aspect may further comprise: providing an ion selective membrane outside the porous framework.

The method of the fourth aspect may further comprise, wherein the sensor chip has a first side having the electrodes and a second side opposite the first side and provides an open liquid junction between the hygroscopic hydrogel electrolyte on the first side of the chip and an external environment on the second side of the chip.

The working electrode may be mounted on the chip or may be separate from the chip.

A further aspect of the invention, comprising: an aromatic epoxy polymer made from one or more monomers having at least one epoxide group; and a chloride salt or silver—silver chloride physically trapped in the aromatic epoxy polymer; wherein on contact with water the aromatic epoxy polymer forms a network of channels for ion exchange. This is due to hydroxylation within the polymer matrix.

This arrangement provides an easily manufactured, and storable, solution that can be used in a variety of applications such as human and animal monitoring, and environmental sensing applications. The network of channels creates a tortuous path through the polymer membrane that limits the loss of ions i.e. Cl⁻ and AgCl₂⁻. The impedance of the membrane is reduced by the inclusion of the salt. This is particularly beneficial to electrochemical measurement systems.

The one or more monomers may have at least one epoxide group from which the aromatic epoxy polymer is made are bis-phenyl monomers, preferably bis-phenyl monomers of general formula I:

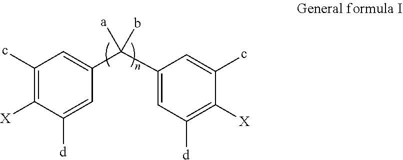

wherein a, b, c and d are H or CH₃;
X is

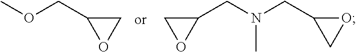

n is 0, 1 or 2,

The aromatic epoxy polymer may be made by reacting the one or more monomers with a hardener, preferably an amine hardener, more preferably an amine hardener of general formula II:

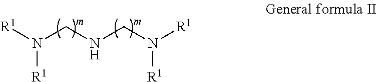

wherein m is 2 to 6, preferably 3 and R¹ is CH₃ or H.

A further aspect provides an electrochemical measurement system comprising: a reference electrode, a working electrode, an electrolyte in contact with the reference electrode and the working electrode, an external fluid environment, and the ion selective membrane between the electrolyte and the external fluid environment.

A further aspect provides an integrated reference electrode comprising: a reference electrode, and the ion selective membrane directly contacting the reference electrode.

This provides a reference electrode that maintains a stable potential in solution, whilst dispensing with the need for an internal electrolyte solution, e.g. in a housing.

A further aspect provides an electrochemical measurement system, comprising: the integrated reference electrode, and a working electrode, wherein the working electrode and the reference electrode both directly contact a measurement environment.

A further aspect provides a method of preparing the ion selective membrane for use in an electrochemical measurement system, comprising: hydrating the ion selective membrane prior to use in the electrochemical measurement system.

The pores and/or channels may comprise a mean diameter of less than 1 micron, preferably less than 500 nm, and more preferably less than 100 nm.

The ion selective membrane may have a thickness of less than 2 mm and/or at least 10 microns.

The ratio of polymer to salt by (pre-polymerised) weight may be less than 5:1, preferably less than 3:1, and more preferably approximately 1:1.

The ratio of monomer to hardener may be 1:1. The ratio of monomer to hardener may be 4:1.

The reference electrode may be a silver—silver chloride electrode.

The reference electrode may comprise a wire, rod, pellet, or a micro-fabricated electrode.

### BRIEF DESCRIPTION OF THE DRAWINGS

Embodiments of the invention will now be described with reference to the accompanying drawings, in which:

FIG. 1A shows a sensor chip in which a reference electrode and a pH sensor are fabricated on a substrate;

FIG. 1B shows a sensor chip in which a reference electrode, a pH sensor, and a dissolved oxygen sensor are fabricated on a substrate;

FIG. 2 shows a process for forming an impregnated porous framework of an integrated reference electrode;

FIG. 3 shows an exploded view of a sensor system according to a first example;

FIG. 4A shows a second example of a sensor system in a dehydrated state;

FIG. 4B shows the second example of the sensor system in a hydrated state;

FIG. 5 shows a comparison of the absorption ratio of the porous framework in different conditions;

FIG. 6 shows part of an electrochemical measurement system or sensor system 1 including a reference electrode 12;

FIG. 7 shows a solid-state integrated reference electrode.

### DETAILED DESCRIPTION OF EMBODIMENT(S)

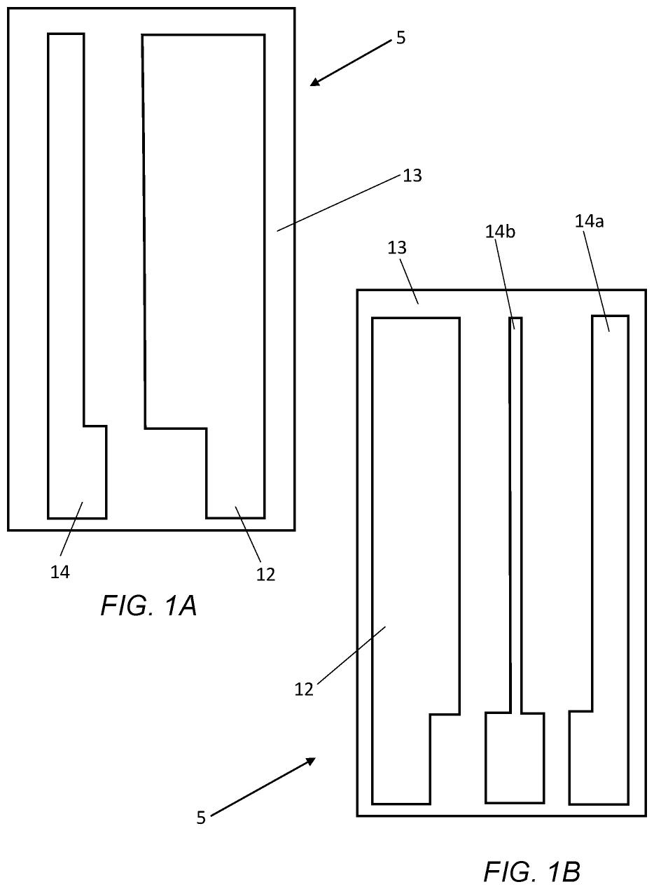

FIG. 1A shows a sensor chip 5. The sensor chip includes an integrated reference electrode 10. An integrated reference electrode is a reference electrode 12 in combination with a hygroscopic hydrogel electrolyte impregnated and contained in a porous framework 21. The integrated reference electrode 10 may comprise a silver—silver chloride (Ag/AgCl) reference electrode 12. The reference electrode 12 may be fabricated on a substrate 13. In alternative examples, the reference electrode 12 may be a standalone reference electrode 12 that is not fabricated on a substrate. The reference electrode 12 may be in any suitable form, such as a wire, rod, or pellet.

The substrate 13 may comprise glass or polymeric material. The reference electrode 12 may be deposited on the substrate 13 using any suitable deposition method, such as a physical, chemical, or electro deposition method. Suitable examples may include screen printing, vacuum evaporation, chemical bath deposition, electro-deposition, and spray pyrolysis.

The reference electrode 12 may be combinable with one or more further working electrodes 14. FIG. 1A shows the substrate 13 including a reference electrode 12 in combination with a pH sensor 14. FIG. 1B shows an alternative sensor chip 5 in which the substrate 13 includes a reference electrode 12 in combination with a pH sensor 14a and a dissolved oxygen sensor 14b. It will be appreciated that the sensor chip 5 may include any number and combination of different sensors 14.

The sensor chip 5 forms part of an electrochemical measurement system that can analyse a sample using the integrated reference electrode 10. Conventional measurement systems suffer from a number of difficulties, such as evaporation and dehydration of the electrolyte solution, as well as difficulties that arise due to the need for sterility 12 used in medical applications. The integrated reference electrode 10 described herein tackles one or more of these difficulties.

The integrated reference electrode 10 includes a reference electrode 12 in combination with a hygroscopic hydrogel electrolyte impregnated and contained in a porous framework 21.

The hygroscopic hydrogel electrolyte is formed by combining a hydrogel monomer 23 with a chloride salt 24, such as potassium chloride or sodium chloride. The hydrogel 23 is preferably chitosan, which is a biocompatible and bactericidal hydrogel with a charged polymer chain structure that provides several tunable properties, including the absorption and retention of liquid (hygroscopicity) and bactericidal properties. Alternative hydrogels 23 will occur to the skilled person including, but not limited to, polymers of acrylamide, alginate, agarose, chitin or other derivatives of chitin.

A hydrogel 23 is a gel in which, in its swollen form, the main constituent is liquid water. A cross-linking agent is added to give the hydrogel its mechanical properties. The quantity of cross-linking agent can be tailored to give particular properties. The hydrogel allows the ions to move freely in an integrated electrode arrangement, thereby creating an ionic interface between the reference electrode and an external environment (e.g. a patient's tissue). Furthermore, hydrogels can be stored dry and (re-) hydrated when required to assume their hydrated form which may include their original or expanded size. Salts such as sodium chloride (NaCl) and potassium chloride (KCl) can be incorporated into the hydrogel to provide a known chloride activity.

The porous framework 21 may be a scaffold, foam or sponge material (noting that there may be some overlap between these terms). Suitable materials for the porous framework include, for example, poly-dimethyl siloxane (PDMS) and poly-urethane (PU). The porous framework 21 forms a porous structure throughout which the hygroscopic hydrogel electrolyte can be impregnated. The porous framework 21 may have a substantially uniform pore size, or may have a distribution of pore sizes.

The porous framework 21 ensures contact between the surface of the reference electrode 12 and the hydrogel electrolyte when the hygroscopic hydrogel electrolyte is in a hydrated state, with that contact being further improved by the swelling of the hydrogel 23 when it absorbs liquid from its surroundings. The porous framework 21 can also maintain its structure when the hydrogel 23 is dehydrated, ensuring that the hydrogel 23 is well supported and well positioned to resume contact with the reference electrode 12 when it is rehydrated. Problems with a loss of ionic contact, and consequently loss of the ionic bridge within the electrochemical cell, are therefore mitigated when the hygroscopic hydrogel electrolyte is hydrated. The hygroscopic hydrogel electrolyte may contact the reference electrode 12 only when hydrated. The hygroscopic hydrogel electrolyte may contact the reference electrode 12, for example the porous framework 28 may be bonded to the surface of the reference electrode 12 so that direct contact is maintained in the hydrated and dehydrated states.

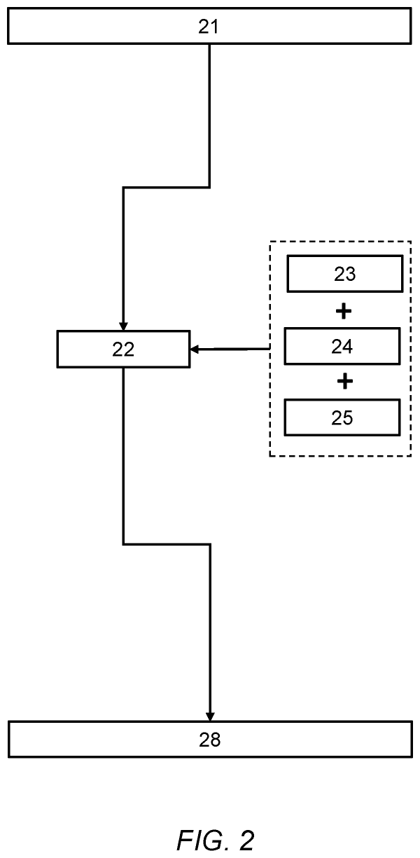

FIG. 2 shows a process of forming the impregnated framework of the integrated reference electrode 10.

A porous framework 21 is provided, such as a foam. Prior to addition of a hygroscopic hydrogel electrolyte solution, the porous framework 21 may undergo a plasma treatment, such as oxygen plasma treatment, to increase the hydrophilicity of the porous framework 21 by oxidizing the surface layer. Alternatively, the porous framework 21 may be inherently hydrophilic or a hydrophilic coating added. This provides a hydrophilic porous framework 22.

The hydrophilic porous framework 22 is then immersed in a mixture of the electrolyte solution (containing a hydrogel monomer 23 and a chloride salt 24) and a crosslinking agent 25, for example a natural crosslinking agent such as genipin or alternatively a synthetic crosslinking agent, in order to impregnate the hydrophilic porous framework 22. The hydrophilic nature of the hydrophilic porous framework 22 facilitates the impregnation of the combined electrolyte solution and polymer/cross-linking 25 mixture into the framework 22.

The hydrogel solution (containing the hydrogel monomer 23, chloride salt 24, and cross linking agent 25), now immersed in the hydrophilic porous framework 22, is then allowed time to crosslink before being retrieved. The time provided for crosslinking can be any duration that allows the desired crosslinking degree to be achieved. In one example of a chitosan-genipin solution, a time period of approximately 72 hours at room temperature is typically provided. In the case of the latter, the retrieved hygroscopic hydrogel within the hydrophilic porous framework can be neutralized using alkaline solutions (i.e. NaOH).

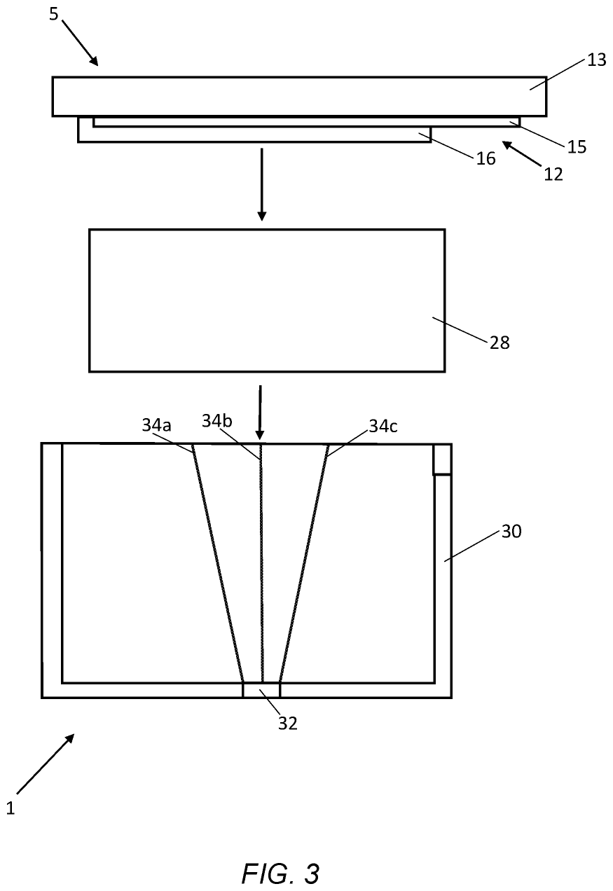

The cured porous framework 28, into which the hydrogel solution is now impregnated, is then dried for storage and later use. Drying can be achieved in any suitable manner, for example air drying or freeze drying. The cured porous framework 28 can be cut to the desired size for an electrode assembly or housing 30, as shown in FIG. 3.

FIG. 3 shows an exploded view of the components of a sensor system 1. As shown, the impregnated cured porous framework 28 is placed into a housing 30. The housing 30 may be formed of, or comprise, any suitable material, for example polypropylene (PP) or poly-methyl methacrylate (PMMA).

At the top of the housing 30, enclosing the housing 30, is a sensor chip 5 including the reference electrode 12. The reference electrode 12 includes a silver—silver chloride coating 16 on a conductive connector 15 that is integrated on the substrate 13. The conductive connector 15 is arranged to conduct the electrons from the reference electrode 12 to an appropriate read-out circuit for analysis and comparative purposes during use. For example, the control of the potential at a working electrode during electrochemical measurements.

At a bottom of the housing 30, opposed to the top of the housing 30, there is a port 32. The port 32 provides a liquid connection between the external measurement environment (i.e. external electrolyte) outside the housing 30 and the porous framework 28 inside the housing 30, thereby allowing the hydrogel 23 of the porous framework 28 to be hydrated (or rehydrated). The port may alternatively be referred to as an aperture.

The port 32 is fluidically connected to conduits 34a, 34b, 34c. FIG. 3 shows the housing 30 includes three conduits 34a, 34b, 34c, although the housing 30 may comprise any number of conduits, for example one conduit, two conduits, four conduits, or more. In some examples, the housing 30 may include no conduits. The conduits 34a, 34b, 34c extend between the top of the housing 30, adjacent a top of the porous framework 28, and a bottom of the housing 30, adjacent a bottom of the porous framework 28.

The conduits 34a, 34b, 34c provide fluid paths through the porous framework 28 so as to facilitate efficient hydration of the hydrogel 23, which is impregnated in the porous framework 28, when fluid is passed through the port 32. In order to facilitate efficient hydration, the conduits 34a, 34b, 34c may have openings only at each end, or may be perforated at specified locations along the length of the conduits 34a, 34b, 34c.

In FIG. 3, the port 32 is shown to form a liquid junction to the external environment through the housing 30. In an alternative example, the port 32 may be formed through the sensor chip 5 itself, such that it forms an open liquid junction between the hygroscopic hydrogel electrolyte on an inner side of the chip and an external environment on the outer side of the chip.

In some examples, in order to ensure direct contact between the reference electrode 12 and the porous framework 28, the porous framework 28 is bonded to the surface of the reference electrode 12, for example using oxygen plasma treatment or surface chemistry (such as silane, epoxy etc).

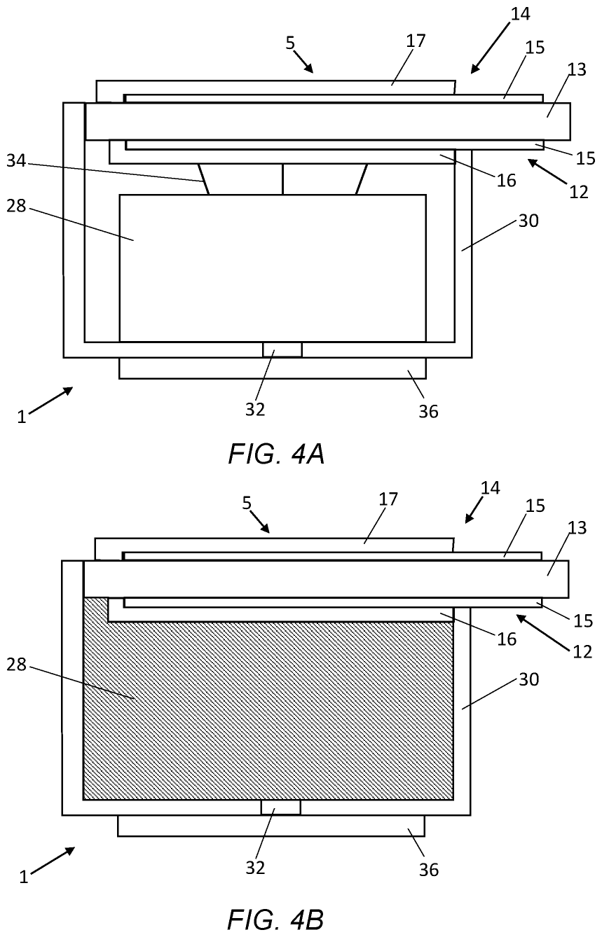

FIGS. 4A and 4B show a sensor system 1 comprising a working electrode 14.

The sensor system 1 may be an implantable sensor system for implanting in a human body. Specifically, the sensor system 1 may be an intrauterine sensor system for implanting into a uterus. In alternative examples, the sensor system 1 may be an implantable sensor system for implanting in an animal body or any other suitable sensor application. The system 1 is particularly advantageous for sensor applications in which it is desirable to provide a stable reference electrode able to maintain potential stability for extended periods.

The working electrode 14 may be a pH sensor, an oxygen sensor, a conductivity sensor, or bio-sensor, or any other suitable sensor to detect a physiological or chemical parameter.

The sensor system 1 may comprise a sensor chip 5, onto which the working electrode 14 (on which the chemical reaction of interest occurs) may be placed. As with the reference electrode 12, the working electrode 14 may include a coating 17 on a conductive connector 15 that is integrated on the substrate 13.

In FIGS. 4A and 4B, the working electrode 14 is mounted on the same sensor chip 5 as the reference electrode 12, and housed within a common housing 20. The sensor system 1 may comprise any quantity of working electrodes, for example one, two, three, four or more.

In alternative examples, the working electrode(s) 14 may be separate to the reference electrode 12, i.e. not mounted to the same sensor chip 5. The working electrode(s) may be mounted on the same side of the sensor chip 5 as the reference electrode 12, or on the opposite side, or separate electrodes 14 may be on each side.

An ion selective membrane 36 may also be provided, as shown in FIGS. 4A and 4B. The membrane 36 may provide a barrier that limits the loss of electrolyte and diffusion of chloride solution from the integrated reference electrode and/or housing 30, whilst also acting as a barrier to biofouling.

The ion selective membrane 36 may be a glass or ceramic frit.

FIG. 4A shows the cured porous framework 28 in a dehydrated state, in which the porous framework 28 does not contact the reference electrode 12, although in alternative examples the porous framework 28 may contact the reference electrode 12 even in the dehydrated state. The porous framework 28 may be completely dehydrated, such that substantially all liquid is removed from the porous framework 28, or may be partially dehydrated such that some relative level of liquid is maintained in the dehydrated state.

In the dehydrated state, the porous framework 28 can be sterilized more efficiently. This is due to the absence (or at least reduction in quantity) of liquid in the porous framework 28, which allows the efficient removal of any toxic compounds (e.g. by gas leaching) that are introduced during gas sterilization. For example, terminal sterilization by EtO can be performed without the risk of toxic residue being retained, and without the risk of the impregnated porous framework 18 being damaged to the extent that loss of contact with the reference electrode 12 is a concern.

Upon contact with a liquid, the hygroscopic hydrogel 23 absorbs the moisture and swells to a hydrated state, as shown in FIG. 4B.

Hydration can be facilitated by any suitable means, for example by placement of droplets of aqueous buffer on the porous framework, immersion in liquid, or exposure to highly humid conditions.

The porous framework 28 may be placed in the housing 30 in the dehydrated state, as it is smaller and easier to fit inside the housing 30 in the dehydrated state. The hydrogel 23 may be subsequently hydrated (e.g. via the port 32), causing the hydrogel 23 to swell and thereby expand itself and the porous framework 28 as a whole so that it expands out towards the reference electrode 12 and the inner walls of the housing 30. It will be clear that the volume of hydrogel 23, and overall size of the porous framework 28, can be tailored to provide a particular performance. Increasing the volume of the hydrogel 23 within the porous framework 28, and increasing the overall size of the porous framework 28, will provide prolonged stability of the reference electrode 12.

This expansion inside the housing 30 ensures a good ionic contact between the hydrated porous framework 28 and the reference electrode 12. As the hydrogel 23 in the porous framework 28 is hygroscopic, the hydrogel 23 will continue to absorb moisture from its surroundings. This prevents the porous framework 28 from dehydrating, and thereby ensures a good contact with the reference electrode 12 is maintained.

The sensor system 1 may comprise conduits 34 that extend from the port 32 through at least a portion of the porous framework 28. The conduits 34, such as those shown in FIG. 4A, may ensure that the liquid entering through the port 32 is efficiently and evenly distributed throughout the porous framework 28. This allows the framework 28 to hydrate more quickly.

The amount of absorption of the hydrogel can be controlled by a number of factors in addition to the amount of water provided to the system. For instance, the degree of crosslinking, the type of polymer used, and the addition of specific hygroscopic components such as divalent cations (e.g. MgCl₂) that further limit electrolyte reabsorption into the surroundings (e.g. the surrounding tissues of a patient). As the hydrogel is hygroscopic, the hydrogel may retain water even at elevated temperatures (e.g. 37 degrees C. and above).

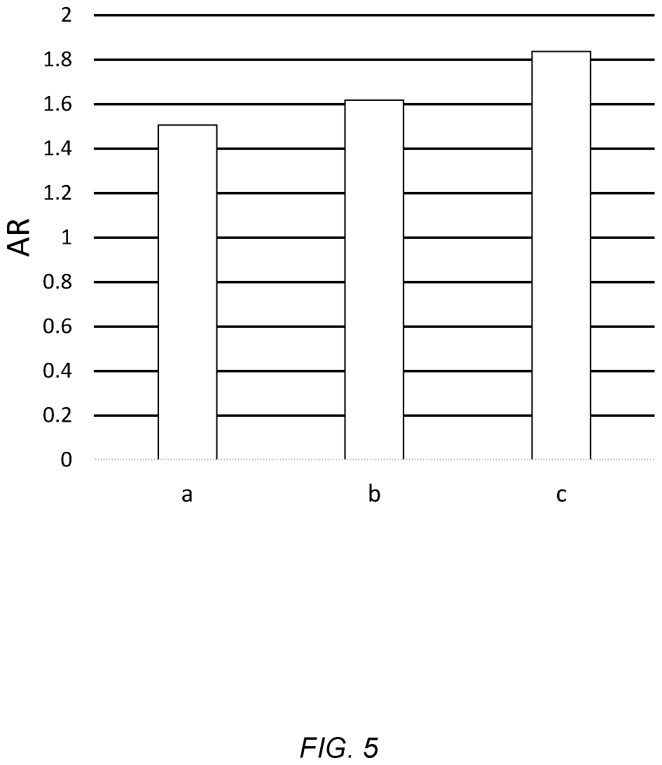

The absorption capabilities of the impregnated porous framework cause the weight of the integrated reference electrode to increase due to the absorption of water by the hygroscopic hydrogel electrolyte impregnated and contained in the porous framework. FIG. 5 shows the absorption ratio (γ-axis), with respect to the dry weight, averaged over five samples of a chitosan-genepin impregnated PDMS foam sample in three different states: (a) a cured state immediately following retrieval from the cross-linking process; (b) when hydrated upon contact with water; and, (c) when incubated in a humid environment at 37° C.

The ion selective membrane 36 may be a glass or ceramic frit.

In an alternative example, the ion selective membrane 26 is an epoxy-salt composite membrane that comprises an aromatic epoxy polymer made from one or more monomers having at least one epoxide group, and a chloride salt or silver—silver chloride physically trapped in the aromatic epoxy polymer. The ion selective membrane 36 is configured to permit ion exchange between an electrolyte within an electrochemical measurement system on one side of the membrane and an external electrolyte on the other side of the membrane.

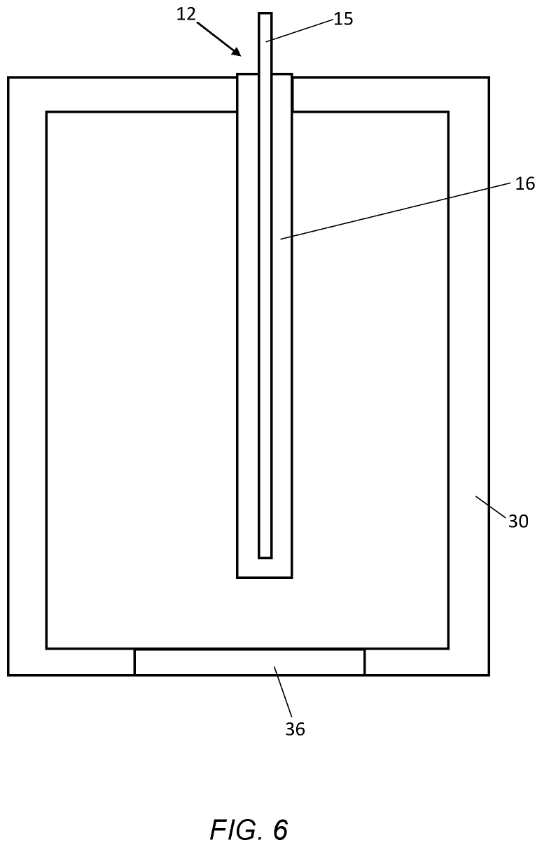

FIG. 6 shows part of an electrochemical measurement system or sensor system 1 including a reference electrode 12. The reference electrode 12 includes a silver conductive connector 15 coated in a layer of silver chloride 16. The combined silver—silver chloride layer is typically formed by electroplating of the conductive connector 15, although other manufacturing techniques are known such as dipping the conductive wire 15 in a silver—silver chloride paste or paint.

The conductive connector 15 may be a wire, rod, pellet, a micro-fabricated electrode, or any other suitable arrangement.

The reference electrode 12 may be housed in a housing 30. The housing 30 is formed of a suitable material for housing the arrangement, such as glass or plastic. The housing 30 may be filled with an electrolyte, such as a chloride salt. The chloride salt may be potassium chloride salt or sodium chloride salt. The salt may have a mean particle size of approximately 0.5 microns diameter.

The housing 30 may include the epoxy-salt composite membrane 36 that forms a barrier between the electrolyte of the system 1 on a first side and an external electrolyte on a second side of the membrane 36 opposite to the first side.

The epoxy-salt composite membrane 36 is created using aromatic epoxy polymers incorporating a salt, such as KCl, NaCl or silver chloride salt. The salt may show limited solubility in water. The membrane 36 may be formed by combining an epoxy monomer with the salt to form a homogeneous mixture, before it is polymerised. The mixture may be degassed prior to application.

It is thought that some of the epoxy polymer bonds are hydrolysed upon contact with water, to leave channels. The water absorption and hydrolysis rate is variable and dependent upon the specific epoxy polymer used. As water penetrates the aromatic epoxy-salt composite, some of the bonds are broken and hydrolysed over time. This creates channels within and/or through the membrane 36 over time. The channels may extend through the membrane 36 from a first side to a second side opposing the first side.

The channels may be permanently open, and subsequently open further upon hydration of the membrane 36. Alternatively, the channels may be closed until the membrane 36 is hydrated, such that when the membrane 36 is hydrated the channels open up.

The thickness of the membrane 36 is chosen based on the desired hydration time of the membrane 36, due to the relationship that exists between the degree of hydrolysation throughout the membrane 36 and the thickness of the membrane 36. The thickness of the membrane 36 may be less than 2 mm. The thickness of the membrane 36 may be less than 1 mm. The thickness of the membrane 36 may be greater than 0.3 mm. The thickness of the membrane 36 may be controlled using various techniques including machining, etching and patterning.

The rate of hydrolisation through the thickness of the membrane 36 may be further altered by creating pores within the membrane during manufacture. For example, pores may be created by the introduction of gas during formation of the membrane 36, or soluble materials, such as salts and sugars, may be added during formation.

As the rate of diffusion is limited by the interfacial area, the size of the pores and channels can be tailored to limit the rate of diffusion of chloride through the membrane 36, thereby maintaining the potential stability of the reference electrode 12 in the sensor system 1 and significantly extending the life-time of the reference electrode 12.

The pores and/or channels may comprise a mean diameter of less than 1 micron when the membrane 36 is hydrated. The pores or channels may comprise a mean diameter of less than 500 nanometers when the membrane 36 is hydrated. In some examples, the mean diameter may be less than 100 nanometers.

During use, the internal electrolyte of the system 1 and external electrolyte of the system to be measured will penetrate the membrane 36 from opposing sides. As hydration occurs from both sides of the membrane 36, an interface will be created between the internal electrolyte and the external electrolyte. The interface is determined by the diameter of the paths created through hydrolisation of the epoxy membrane and may be controlled by the salt additives or manufacturing procedure. As the size of the interface is small and the path length is long, the diffusion of the chloride ion which determines the reference electrode potential stability is retarded.

The rate of diffusion may be further limited by the addition of the salt (e.g. a chloride salt and/or silver salt), as the inclusion of the salt helps to saturate the internal channels and pores when they come into contact with the penetrating water. This acts as a buffer inside the channels, as the water within said channels is constantly saturated and so no further chloride will be dissolved and consequently lost to the external environment. An added benefit to the addition of this salt is that the impedance of the reference electrode 12 is reduced due to the high conductivity of the electrolyte solution, which is a particularly important parameter in electrochemical measurement systems.

The membrane 36 can be produced using simple and cheap manufacturing techniques, thereby reducing the cost of materials and subsequent integration into the reference electrode assembly. The membrane 36 may be formed prior to integration into an electrochemical measurement system. The membrane 36 may be inserted into and/or over an aperture in a reference electrode housing 30. The membrane 36 may be formed in-situ into an aperture in a housing 30.

The ion selective membrane 36 may form part of a larger membrane (not shown), such that only part of the membrane is ion selective.

The sensor system 1 may be an implantable sensor system for implanting in a human or animal body. The sensor system 1 may be an intrauterine sensor system for implanting into a uterus. The system 1 is particularly advantageous for sensor applications in which it is desirable to provide a stable reference electrode able to maintain calibration for extended periods.

The membrane 36 is particularly advantageous when used in combination with the integrated reference electrode 10 previously discussed in relation to FIGS. 3 to 4, as the small size of the membrane 36 and the wetting properties of the impregnated porous framework 28 maintain the internal electrolyte concentration and the electrochemical connection to the reference electrode 12. The membrane 36 can be easily manufactured and stored, as well as used in a variety of environmental sensing applications, such as human and animal monitoring.

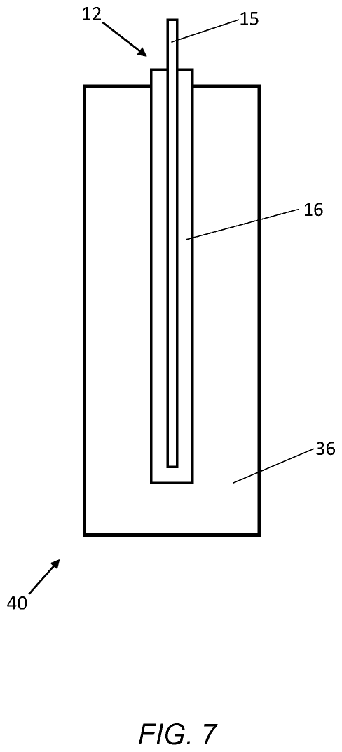

FIG. 7 shows an integrated reference electrode 40 comprising a reference electrode 12 and the epoxy-salt composite membrane 36. The ion selective membrane 36 directly contacts the reference electrode 12, such that the integrated reference electrode 40 is a solid-state integrated reference electrode. The ion selective membrane 36 may substantially envelope the reference electrode 12 so as to form an ion selective membrane 36 that separates the reference electrode 12 from an external environment.

The reference electrode 12 may be a silver—silver chloride electrode. The reference electrode 12 may comprise a conductive connector 15 coated in a coating 16. The coating may be a silver chloride coating. The conductive connector 15 may be silver. The conductive connector 15 may be a wire, rod, pellet, a micro-fabricated electrode, or any other suitable arrangement.

In a solid state integrated reference electrode arrangement, the membrane 36 contacts the coating 16 of the reference electrode directly. This dispenses with the need for an internal electrolyte solution. The membrane 36 allows ion exchange through itself, whilst preventing or retarding transport of the external electrolyte solution through the membrane 36 to the reference electrode. A solid-state electrode 40 has many advantages over conventional liquid-filled electrodes, including increased compactness, as well as easier storage, transport, miniaturization, sterilization, and fabrication.

The integrated reference electrode 40 may form part of a sensor system. The sensor system 1 may comprise one or more working electrodes 14.

The sensor system 1 may be an implantable sensor system for implanting in a human or animal body. The sensor system 1 may be an intrauterine sensor system for implanting into a uterus. The system 1 is particularly advantageous for sensor applications in which it is desirable to provide a stable reference electrode able to maintain calibration for extended periods.

Where the word ‘or’ appears this is to be construed to mean ‘and/or’ such that items referred to are not necessarily mutually exclusive and may be used in any appropriate combination.

Although the invention has been described above with reference to one or more preferred embodiments, it will be appreciated that various changes or modifications may be made without departing from the scope of the invention as defined in the appended claims.

1. An integrated reference electrode for use in an electrochemical measurement system, comprising:
a reference electrode in combination with a hygroscopic hydrogel electrolyte impregnated and contained in a porous framework, wherein the hygroscopic hydrogel electrolyte is adapted to contact the reference electrode when in a hydrated state.

2. The integrated reference electrode of claim 1, wherein the reference electrode is a silver—silver chloride electrode.

3. The integrated reference electrode of claim 1, wherein the porous framework is a scaffold, foam or sponge material.

4. The integrated reference electrode of claim 1, wherein the porous framework includes one or more of poly-urethane (PU) or poly-dimethyl siloxane (PDMS).

5. The integrated reference electrode of claim 1, wherein the hygroscopic hydrogel electrolyte is biocompatible and/or bactericidal.

6. The integrated reference electrode of claim 1, wherein the hygroscopic hydrogel electrolyte includes a chloride salt.

7. The integrated reference electrode of claim 1, wherein the hygroscopic hydrogel electrolyte includes one or more from the group: acrylamide, alginate, agarose, chitin, chitosan or other chitin derivatives.

8. The integrated reference electrode of claim 1, wherein the porous framework is hydrophilic, or has a hydrophilic coating.

9. The integrated reference electrode of claim 1, wherein the reference electrode comprises a wire, rod, pellet, or a micro-fabricated electrode.

10. The integrated reference electrode of claim 1, further comprising an ion selective membrane outside the porous framework.

11. The integrated reference electrode of claim 1, wherein the reference electrode is bonded to the porous framework.

12. A sensor system for use in an electrochemical measurement system, the sensor system comprising:
an integrated reference electrode including a reference electrode in combination with a hygroscopic hydrogel electrolyte impregnated and contained in a porous framework, and
at least one working electrode,
wherein the hygroscopic hydrogel electrolyte is adapted to contact the reference electrode when in a hydrated state.

13. The sensor system of claim 12, further comprising a sensor chip having the reference electrode, wherein the integrated reference electrode and the sensor chip are housed within a common housing.

14. The sensor system of claim 13, wherein the at least one working electrode is in contact with the hygroscopic hydrogel electrolyte.

15. The sensor system of claim 13, wherein the chip has a first side having the reference electrode and a second side opposite the first side and provides an open liquid junction between the hygroscopic hydrogel electrolyte on the first side of the chip and an external environment on the second side of the chip.

16. The sensor system of claim 12, wherein the sensor system is an implantable sensor system for implanting in a human or animal body.

17. A method of manufacturing an integrated reference electrode for use in an electrochemical measurement system so as to provide a reference potential, the method comprising:
preparing a hygroscopic hydrogel electrolyte impregnated and contained in a porous framework;
providing a reference electrode; and
placing the reference electrode and the porous framework having the hygroscopic hydrogel electrolyte therein within a common housing, such that the hygroscopic hydrogel electrolyte is able to contact the surface of the reference electrode when in a hydrated state.

18-21. (canceled)

22. A method of manufacturing a sensor system, comprising:
manufacturing an integrated reference electrode according to the method of claim 17;
providing a sensor chip having the reference electrode and at least one working electrode,
wherein the integrated reference electrode and the sensor chip are housed within the common housing, and the at least one working electrode is in contact with the reference electrode.

23. (canceled)

24. An ion selective membrane, comprising:
an aromatic epoxy polymer made from one or more monomers having at least one epoxide group; and
a chloride salt or silver—silver chloride physically trapped in the aromatic epoxy polymer;
wherein on contact with water the aromatic epoxy polymer forms a network of channels for ion exchange.

25-29. (canceled)

30. An electrochemical measurement system comprising:
a reference electrode,
a working electrode,
an electrolyte in contact with the reference electrode and the working electrode,
an external fluid environment, and
an ion selective membrane between the electrolyte and the external fluid environment,
wherein the ion selective membrane includes an aromatic epoxy polymer made from one or more monomers having at least one epoxide group, and a chloride salt or silver—silver chloride physically trapped in the aromatic epoxy polymer,
wherein on contact with water the aromatic epoxy polymer forms a network of channels for ion exchange.

31. An integrated reference electrode system comprising:
a reference electrode,
an ion selective membrane directly contacting the reference electrode,
wherein the ion selective membrane includes an aromatic epoxy polymer made from one or more monomers having at least one epoxide group, and a chloride salt or silver—silver chloride physically trapped in the aromatic epoxy polymer,
wherein on contact with water the aromatic epoxy polymer forms a network of channels for ion exchange.

32. An electrochemical measurement system, comprising:
a integrated reference electrode; and
a working electrode,
wherein the integrated reference electrode includes a reference electrode and an ion selective membrane directly contacting the reference electrode, wherein the ion selective membrane includes an aromatic epoxy polymer made from one or more monomers having at least one epoxide group, and a chloride salt or silver—silver chloride physically trapped in the aromatic epoxy polymer, and wherein on contact with water the aromatic epoxy polymer forms a network of channels for ion exchange, and
wherein the working electrode and the reference electrode both directly contact a measurement environment.

33. (canceled)

34. (canceled)

35. A method of preparing the ion selective membrane of claim 24 use in an electrochemical measurement system, comprising:
hydrating the ion selective membrane prior to use in the electrochemical measurement system.
